(19) United States
(12) Patent Application Publication
(10) Pub. No.: US 2023/0347092 A1
(43) Pub. Date: Nov. 2, 2023

(54) MOBILE RESPIRATOR

(71) Applicant: GrowTrend Biomedical Co., Ltd., HSINCHU CITY (TW)

(72) Inventors: Pei-Yin OU, New Taipei City (TW); Ching-Liang YU, Taoyuan City (TW); Yun-Yueh LIU, Miaoli City (TW); Neng Yu PAN, New Taipei City (TW)

(73) Assignee: GrowTrend Biomedical Co., Ltd., HSINCHU CITY (TW)

(21) Appl. No.: 18/135,296

(22) Filed: Apr. 17, 2023

(30) Foreign Application Priority Data
Apr. 28, 2022 (TW) 111204421

(51) Int. Cl. A61M 16/10; A62B 7/10; A62B 9/00; A61M 16/06

(52) U.S. Cl. CPC A61M 16/1045; A62B 7/10; A62B 9/003; A61M 16/06; A61M 2210/0618

(57) ABSTRACT

A mobile respirator has a respiratory device, a pipe, a breathing component, and a power bank. The pipe has two opposite ends respectively connected to the respiratory device and the breathing component, and the power bank is electrically connected to the respiratory device. A user can place the power bank and the respiratory device inside a backpack, and the pipe can be outstretched from the backpack. Thereby, the user can carry the backpack and wear the breathing component, so the user may breathe with assistance of the mobile respirator. By the power bank supplying electricity for the respiratory device, the mobile respirator is mobile outdoors and solves the trouble of dyspnea while exercising or staying outdoors.

### BACKGROUND OF THE INVENTION

### 1. Field of the Invention

The present invention relates to a respirator, and particularly to a mobile respirator.

### 2. Description of Related Art

Since nowadays air is seriously polluted, many patients having respiratory diseases may suffer allergy or other symptoms no matter indoors or outdoors. Thereby, many respirators and related products are provided on the market for patients to use during home sleep. The conventional respirator has a great volume and needs to be fixed on a platform and plugged in for use. Moreover, the conventional respirator is connected to an extra heating humidifier having a roughly same volume with the conventional respirator, which prevents the respiratory tract of the patient from dryness.

However, since the conventional respirator needs to be assembled and plugged in, the conventional respirator is limited for the patient to use only on the bed or in sleep. The conventional respirator is difficult to move with the patient. Although part of the conventional respirators allow the patient to use in the car or taking public transportations, each one of these conventional respirators still has to operate with the electricity provided by a power supply on the transportation.

To overcome the shortcomings of the conventional respirator, the present invention tends to provide a mobile respirator to mitigate or obviate the aforementioned problems.

### SUMMARY OF THE INVENTION

The main objective of the present invention is to provide a mobile respirator that has a respiratory device electrically connected to a power bank and is thus mobile.

The mobile respirator has a respiratory device, a pipe, a breathing component, and a power bank. The respiratory device has an inlet and an outlet fluidly communicating with an interior of the respiratory device. The pipe has two opposite ends, and one of the two opposite ends of the pipe is connected to the outlet of the respiratory device. The breathing component has a connecting tube connected to the other one of the two opposite ends of the pipe. The power bank is electrically connected to the respiratory device.

Other objectives, advantages and novel features of the invention will become more apparent from the following detailed description when taken in conjunction with the accompanying drawings.

### BRIEF DESCRIPTION OF THE DRAWINGS

FIG. 1 is a front view of a preferred embodiment of a mobile respirator in accordance with the present invention omitting part of a pipe;

FIG. 2 is an operational view of the mobile respirator in FIG. 1; and

FIG. 3 is a front view of another embodiment of a mobile respirator in accordance with the present invention omitting part of the pipe.

### DETAILED DESCRIPTION OF THE PREFERRED EMBODIMENT

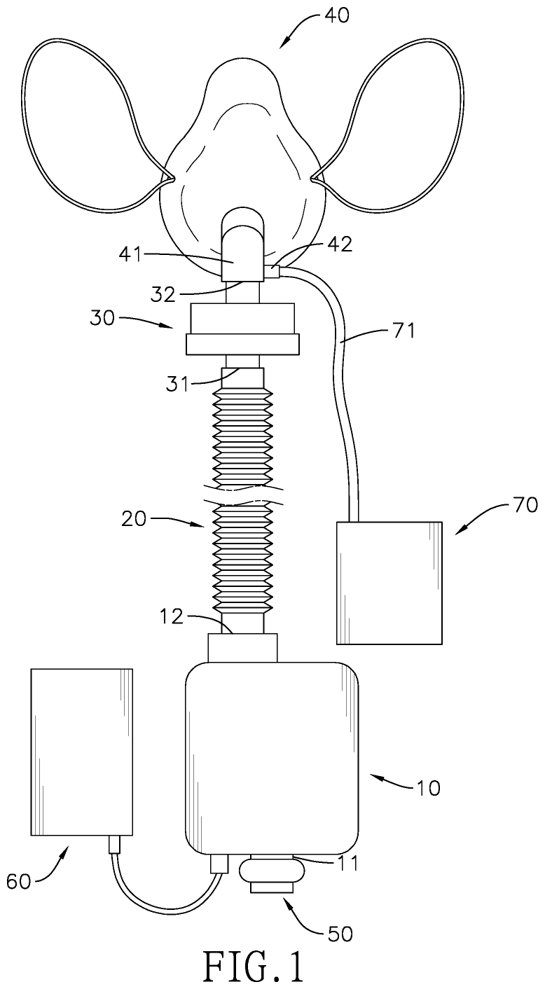

With reference to FIG. 1, a mobile respirator of a preferred embodiment in accordance with the present invention has a respiratory device 10, a pipe 20, a heat and moisture exchange filter (HMEF/FHME) 30, a breathing component 40, a filtering component 50, a power bank 60, and an air supplier 70.

With reference to FIG. 1, the respiratory device 10 has an inlet 11 and an outlet 12, and the inlet 11 and the outlet 12 fluidly communicate with an interior of the respiratory device 10. The respiratory device 10 is an auxiliary of an organ and is configured to generate positive pressure for assisting a patient in improving efficiency of gas exchange in the respiratory system of the patient. The respiratory device 10 has a portable construction such as a respiratory device in a conventional micro respirator. The respiratory device 10 is conventional and is thus not described in detail.

With reference to FIG. 1, the pipe 20 has two opposite ends, and one of the two opposite ends of the pipe 20 is connected to the outlet 12 of the respiratory device 10 to make the pipe 20 fluidly communicate with the respiratory device 10. The pipe 20 can be a pipe having a fixed shape or a flexible pipe such as a hosepipe. The pipe 20 is conventional and is thus not described in detail.

With reference to FIG. 1, the heat and moisture exchange filter (HMEF/FHME) 30 has an inlet opening 31 and an outlet opening 32, and the inlet opening 31 and the outlet opening 32 fluidly communicate with an interior of the heat and moisture exchange filter 30. The inlet opening 31 is connected to the other one of the two opposite ends of the pipe 20. The heat and moisture exchange filter 30 is a bacteria filter configured to heat and humidify air; moisture and heat exhaled by the patient are condensed and kept in the heat and moisture exchange filter 30 for heating and humidifying air later inhaled by the patient. Thereby, moisture of the air exhaled can be kept for increasing humidity of the air inhaled and prevent the air inhaled from dryness. The heat and moisture exchange filter 30 is a conventional component and is thus not described in detail. In other embodiments, the mobile respirator may omit the heat and moisture exchange filter 30, which is not limited to the preferred embodiment.

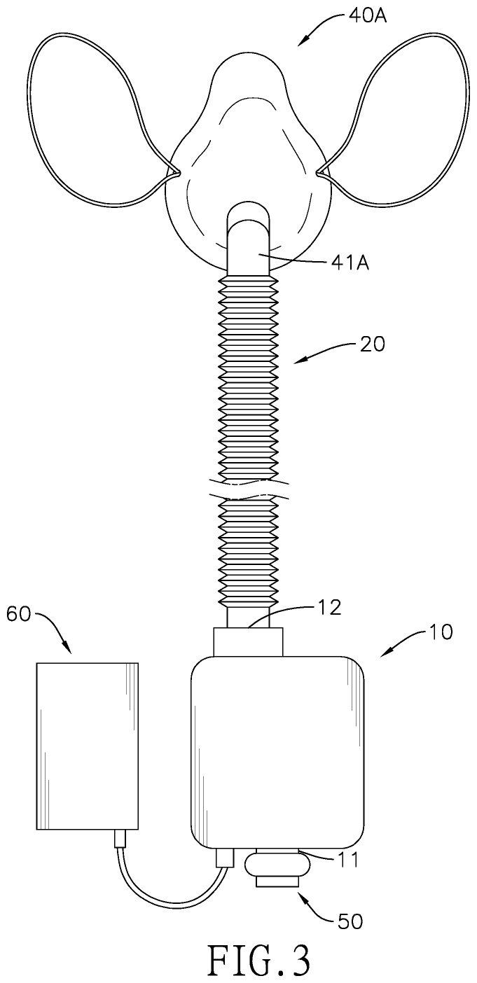

The breathing component 40 has a connecting tube 41 disposed on a side of the breathing component 40 and a branch tube 42. The branch tube 42 is formed on a surrounding wall of the connecting tube 41 and fluidly communicates with the connecting tube 41. The connecting tube 41 of the breathing component 40 is connected to the outlet opening 32 of the heat and moisture exchange filter 30. In the preferred embodiment, the breathing component 40 is a face mask, but in other embodiments, the breathing component 40 may be a nasal cannula or a nasal mask which allows air supplied from the respiratory device 10 to enter the nasal cavity of the patient. Configuration of the breathing component 40 is not limited to the preferred embodiment. Otherwise, in other embodiments, the breathing component 40 may omit the branch tube 42. With reference to FIG. 3, in a mobile respirator of an embodiment omitting the heat and moisture exchange filter 30, the connecting tube 41 of the breathing component 40 is directly connected to the pipe 20.

With reference to FIG. 1, the filtering component 50 is disposed to the inlet 11 of the respiratory device 10 for filtering pollutants such as dust. The filtering component 50 is conventional and is thus not described in detail. In other embodiment, the mobile respirator may omit the filtering component 50, which is not limited to the preferred embodiment.

With reference to FIG. 1, the power bank 60 is electrically connected to the respiratory device 10. The power bank 60 is conventional and is thus not described in detail.

With reference to FIG. 1, the air supplier 70 has a supplying pipe 71, and an end of the supplying pipe 71 away from the air supplier 70 is connected to the branch tube 42 of the breathing component 40. In the preferred embodiment, the air supplier 70 is a portable oxygen concentrator. In another embodiment, the air supplier 70 may supply another gas or atomized medication, and moreover, in other embodiments, the mobile respirator may omit the air supplier 70; the above configuration is not limited to the preferred embodiment.

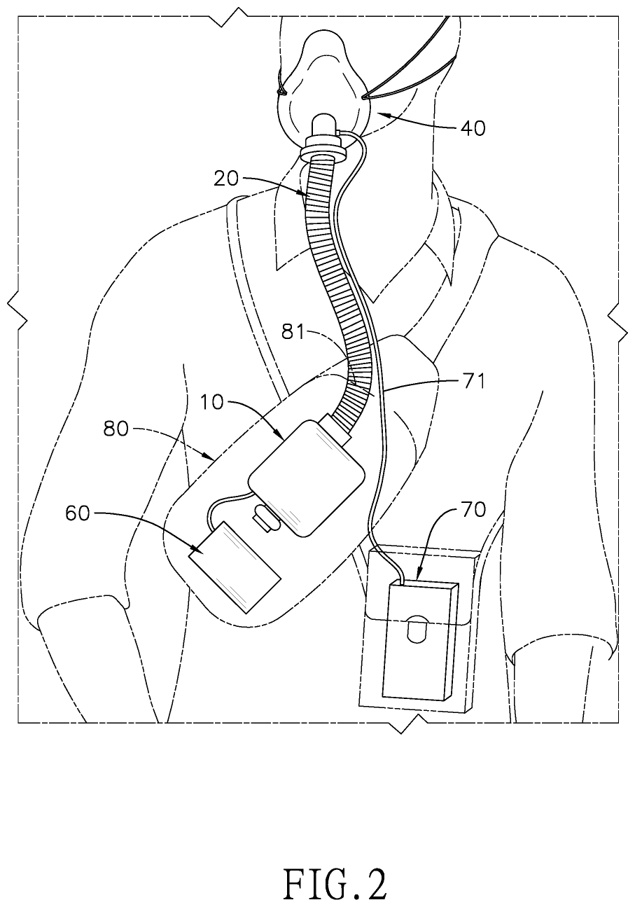

With reference to FIG. 2, in use of the mobile respirator of the preferred embodiment, the respiratory device 10, the power bank 60, and the air supplier 70 may be placed inside a backpack 80 having an opening 81 and multiple air openings (not shown in Figure); the pipe 20 and the supplying pipe 71 are outstretched from the opening 81 of the backpack 80. Thereby, a user can carry the backpack 80 and wear the breathing component 40, so the user may breathe with assistance of the mobile respirator while moving. The power bank 60 supplies electricity for the respiratory device 10, so the mobile respirator can be mobile outdoors and can solve the trouble of dyspnea while the user is exercising or staying outdoors. Compared to the conventional respirator only available at a fixed position, the mobile respirator of the present invention improves convenience in use.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A mobile respirator comprising:
a respiratory device having an inlet and an outlet fluidly communicating with an interior of the respiratory device; and
a pipe having
two opposite ends, and one of the two opposite ends of the pipe connected to the outlet of the respiratory device;
a breathing component having
a connecting tube connected to the other one of the two opposite ends of the pipe; and
a power bank electrically connected to the respiratory device.

2. The mobile respirator as claimed in claim 1, wherein
the mobile respirator comprises an air supplier having a supplying pipe;
the breathing component has a branch tube formed on and fluidly communicating with the connecting tube; and
an end of the supplying pipe away from the air supplier is connected to the branch tube.

3. The mobile respirator as claimed in claim 1, wherein
the mobile respirator comprises a heat and moisture exchange filter;
the heat and moisture exchange filter has an inlet opening and an outlet opening fluidly communicating with an interior of the heat and moisture exchange filter;
the inlet opening of the heat and moisture exchange filter is connected to the other one of the two opposite ends of the pipe; and
the outlet opening of the heat and moisture exchange filter is connected to the connecting tube.

4. The mobile respirator as claimed in claim 2, wherein
the mobile respirator comprises a heat and moisture exchange filter;
the heat and moisture exchange filter has an inlet opening and an outlet opening fluidly communicating with an interior of the heat and moisture exchange filter;
the inlet opening of the heat and moisture exchange filter is connected to the other one of the two opposite ends of the pipe; and
the outlet opening of the heat and moisture exchange filter is connected to the connecting tube.

5. The mobile respirator as claimed in claim 3, wherein the mobile respirator comprises a filtering component disposed to the inlet of the respiratory device.

6. The mobile respirator as claimed in claim 4, wherein the mobile respirator comprises a filtering component disposed to the inlet of the respiratory device.

7. The mobile respirator as claimed in claim 5, wherein the breathing component is a face mask.

8. The mobile respirator as claimed in claim 6, wherein the breathing component is a face mask.

9. The mobile respirator as claimed in claim 5, wherein the breathing component is a nasal cannula.

10. The mobile respirator as claimed in claim 6, wherein the breathing component is a nasal cannula.

11. The mobile respirator as claimed in claim 5, wherein the breathing component is a nasal mask.

12. The mobile respirator as claimed in claim 6, wherein the breathing component is a nasal mask.
